(19) United States
(12) Patent Application Publication
(10) Pub. No.: US 2002/0042928 A1
(43) Pub. Date: Apr. 11, 2002

(54) Methods for maximizing expression of transgenic traits in autopolyploid plants

(76) Inventors: Mark H. McCaslin, Prior Lake, MN (US); Stephen J. Temple, Onalaska, WI (US); Jessica E. Tofte, Prior Lake, MN (US)

Correspondence Address: Raymund F. Eich, Williams, Morgan & Amerson, P.C., 7676 Hillmont, Suite 250, Houston, TX, 77040, US

(21) Appl. No.: 09/900,314

(22) Filed: Jul. 6, 2001

Related U.S. Application Data
(60) Provisional application No. 60/217,470, filed on Jul. 11, 2000.

(51) Int. Cl. A01H 5/00; A01H 1/00; A01H 5/10

(52) U.S. Cl. 800/260; 800/278; 800/266; 800/267; 800/300; 800/302

(57) ABSTRACT

A process for maximizing the transmission of transgenic traits in autopolyploid transgenic plants is provided. According to the process, a plurality of autopolyploid transgenic plant lines, each having a recombinant transgene incorporated into a different region of the plant genome, and each exhibiting a transgenic trait attributable to the transgene, are intercrossed to obtain progeny plants that are trihomogenic and/or tetrahomogenic for the transgenes. These plants are advantageously used in the production of synthetic generations of transgenic plants in which a very high percentage of plants exhibit the transgenic trait. The process affords a significant reduction in resources required to achieve high transmission of transgenic traits in autotetraploid plants and reduces the risks associated with inbreeding, genetic drift, or gene silencing.

The present application claims priority from U.S. Provisional Patent Application 60/217,470, filed Jul. 11, 2000.

### BACKGROUND OF THE INVENTION

1. Field of the Invention

The present invention relates generally to genetically modified plants, and, more particularly, to methods for achieving highly efficient transmission of transgenic traits in autopolyploid crops.

2. Background of the Invention

Crop and plant improvements have traditionally depended on selective breeding of plants with desirable characteristics. Initial breeding success was probably accidental, resulting from observation of a plant with desirable characteristics, and use of that plant to propagate the next generation. However, because such plants had within them heterogeneous genetic complements, it was unlikely that progeny identical to the parent(s) with the desirable traits would emerge. Nonetheless, advances in controlled breeding have resulted from both increasing knowledge of the genetic mechanisms operative in hereditary transmission, and by empirical observations of the results of making various parental plant crosses.

Recent advances in molecular biology have dramatically expanded man's ability to manipulate the germplasm of animals and plants. Genes controlling specific phenotypes, for example specific polypeptides that confer antibiotic or herbicide resistance, have been identified and isolated. Even more important has been the ability to manipulate the genes which have been isolated from one organism and to introduce them into another organism by a process known as transformation. These introduced genes are generally referred to as transgenes and the organisms into which the genes are introduced are called transgenic organisms. Genetic transformation may be accomplished even where the recipient organism is from a different phylum, genus or species from that which donated the gene (i.e., heterologous transformation).

Most genetically modified crop cultivars carry a transgene from a single transgenic event such that the transgene is present at a single location within the plant's genomic DNA. High trait purity (i.e. a high percentage of the plants expressing the transgenic phenotype) may be a desired feature of a transgenic cultivar (e.g. transgenic cultivars with herbicide resistance). After backcrossing T₀ transgenic plants to superior agronomic types, high trait purity in a diploid plant species is easily achieved with a single transgenic event via selection and inbreeding to homozygosity. These homozygous transgenic lines can be used as parents in the production of F1 hybrids or as cultivars. In both of these examples, 100% of the plants in the resulting transgenic varieties will have the transgenic phenotype. From a commercial standpoint, it is very important to have the ability to efficiently produce large numbers of transgenic plants in which this high level of transmission of a transgenic trait can be achieved.

### DEFICIENCIES IN THE PRIOR ART

In contrast to diploid plant species, very high levels of transmission of a transgenic trait (for example, greater than about 90% or 95% of plants in a variety having the transgenic phenotype) in autopolyploid plants requires the duplex, triplex, or quadriplex condition at the transgenic locus. The complexities of autotetraploid genetics makes high transgene transmission (i.e. trait purity) both time and labor intensive, and subjects the resultant population to possible inbreeding depression and/or genetic drift.

When using transgenic plants having a single transgenic event for the commercial production of an autotetraploid cultivar, selfing and/or recurrent phenotypic selection followed by one or two generations of progeny testing is required to produce and identify the desired plants that are duplex, triplex and/or quadriplex for the dominant gene at the transgenic locus. The drawbacks of this approach include:

1) Large initial populations are required because of the low frequency of duplex, triplex, or quadriplex individuals. After one cycle of phenotypic recurrent selection (PRS1) for the transgenic phenotype 33% of the individuals are duplex for the dominant transgenic allele. After three cycles of phenotypic recurrent selection (PRS3) for the transgenic phenotype, 15% of the individuals are triplex and 2% are quadriplex for the dominant transgenic allele. At the present time there is no laboratory test that can precisely and accurately distinguish between plants in segregating populations for individuals with varying doses of a transgene at a single locus (e.g. Axxx—simplex vs AAxx—duplex vs AAAx—triplex, etc.) Therefore, progeny testing is required to identify and discriminate between these multiple genotypes that share the same phenotype (e.g. herbicide tolerance). For example, in order to identify a minimum of 50 triplex/quadriplex plants in a PRS3 breeding population, approximately 300 plants would need to be test-crossed and progeny tested. The cost of progeny testing combined with the low frequency of the desired genotype make this procedure very resource intensive;

2) There is a significant risk of inbreeding depression during the selfing and selection program that is required to increase the frequency of the transgene. This is a very likely problem in the generation of triplex and/or quadriplex genotypes. Both selfing and multiple cycles of recurrent selection narrow the germplasm base and introduce increased inbreeding within populations. Inbreeding in autotetraploid populations commonly results in decreased vegetative vigor and lower seed yield;

3) There is a risk of genetic drift due to small sample sizes at various stages of the selection program. Multiple generations of selection in an autotetraploid may lead to genetic drift, resulting in selected populations that are different than the unselected parent. The progeny testing program also presents risk of genetic drift if the number of selected (i.e. duplex/triplex/quadriplex plants) individuals is less than 75-100. The high cost of progeny testing will likely limit the number of selected individuals used as parents in developing populations with high trait purity;

4) The time and labor required for such an approach will very likely limit the number of genetic backgrounds commercialized with a given transgenic trait; and,

5) The time, labor and greenhouse space required for progeny testing are resource intensive.

Most plant breeding programs require regular introgression of new germplasm (i.e. genetic variation) into the program. The new germplasm serves as a source of new gene/traits, or new combinations of genes/traits for the breeding program (e.g., source of disease or insect resistance genes). Crossing of triallelic or tetrallelic transgenic plants to new sources of non-transgenic germplasm will need to be followed by the resource intensive, multiple-year breeding program outlined above to develop new lines/populations that combine the traits from the new germplasm with high trait purity of the transgenic trait. It would be desirable to have an alternative, simpler breeding program to produce such results.

### SUMMARY OF THE INVENTION

The present invention overcomes or substantially minimizes the effects of the aforementioned problems by using transgenic plants that have multiple independent transgenic events to achieve high levels of transmission of a transgenic trait in autopolyploid plants.

The use of the present invention represents a significant reduction in resources required to achieve high transmission of a transgenic trait in an autopolyploid, e.g. an autotetraploid, population. When high trait purity is desired (e.g. >95% of the plants in a cultivar with the transgenic phenotype) this invention, when compared with a conventional product development strategy using a single transgenic event, significantly decreases the time and cost of product development by employing a product development program using multiple transgenic events and molecular markers (i.e. event specific PCR) to identify segregating plants containing one or more copies of the transgene at multiple independent loci. The invention may also reduce risk by decreasing the likelihood of inbreeding, genetic drift, or gene silencing during transgenic product development.

Therefore, according to one aspect of the present invention, a method is provided for achieving high levels of transmission of a transgenic trait in an autopolyploid, e.g. an autotetraploid, crop.

The method involves the use of multiple, e.g. two, three or four, autopolyploid transgenic plant lines, in which each of the plant lines has one or more copies of a recombinant transgene incorporated into a different region of the plant genome. By “different region” is meant a region which, in the other plant lines, has not received a transgene. Each of the plant lines exhibits a transgenic trait, most typically the same or very similar trait, attributed to the transgene. These transgenic plant lines are intercrossed for one or more generations in order to obtain plants that are multihomogenic. Multihomogenic plants can be identified by the use of various molecular techniques, such as event specific PCR.

The novel term “multihomogenic” is used here to describe plants with one or more copies of the same or similar transgene at each of multiple loci. No nomenclature is currently available to describe this condition. As used herein, dihomogenic refers to genotypes with one or more copies of the transgene at each of two independent loci; trihomogenic refers to genotypes with one or more copies of the transgene at each of three independent loci; and tetrahomogenic refers to genotypes with one or more copies of the transgene at each of four independent loci; etc. The numerals preceding the multihomogenic status describe the number of transgenic alleles at each locus. For example, 1,1 dihomogenic describes a genotype that is simplex (e.g. Axxx, Byyy) for the transgene at both the A and B loci. Similarly, 1,2,2 trihomogenic describes a genotype that is simplex for the transgene at the A locus, and duplex for the transgene at the B and C loci (e.g. AxxxBByyCCzz). Event specific PCR fingerprints can be used to identify the multihomogenic plants in segregating populations.

These multihomogenic (e.g. dihomogenic, trihomogenic, tetrahomogenic, etc.) plants are then intercrossed to produce a first synthetic generation of plants. Preferably greater than 90%, more preferably greater than 95%, and most preferably greater than 97% of the first generation of plants exhibit the transgenic phenotype. The first synthetic generation of plants can be intercrossed to produce a second synthetic generation of plants. Preferably greater than 90%, more preferably greater than 95%, and most preferably greater than 97% of the second generation of plants exhibit the transgenic phenotype. The second synthetic generation of plants can be intercrossed to produce a third synthetic generation of plants. Preferably greater than 90%, more preferably greater than 95%, and most preferably greater than 97% of the third generation of plants exhibit the transgenic phenotype.

Table 1 summarizes the phenotypic analysis of progeny resulting from the intercross of various alfalfa genotypes carrying a Roundup Ready™ (RR) transgene. The expected percentages of genotypes carrying the transgene were calculated according to the Mendelian model.

TABLE 1

| | Expected % RR | Actual % RR | No. of Plants |
|---|---|---|---|
| Genotype | Progeny | Progeny | Tested |
| simplex ((Aaaa) × | 75.0 | 74.2 | 1197 |
| (Aaaa)) | | | |
| 1,1 dihomogenic | 93.7 | 92.0 | 2170 |
| ((AaaaBbbb) × | | | |
| (AaaaBbbb)) | | | |

It will be possible to increase trait purity of multihomogenic populations by combining the methods outlined above (and described in more detail below) with cycles of phenotypic or genotypic recurrent selection for the desired transgenic phenotype or genotype.

According to another aspect of the present invention, introgression of new germplasm into a transgenic breeding program can be rapidly and efficiently achieved. The present invention relates to a method for introgressing non-transgenic germplasm into a transgenic autopolyploid crop, comprising: providing one or more donor parents that are multihomogenic for the transgene; crossing the one or more donor parents to one or more non-transgenic parent plants comprising germplasm containing at least one desirable trait not present in the donor parent, to yield progeny plants, wherein at least one of the progeny plants are multihomogenic for the transgene; identifying progeny plants which are multihomogenic for the transgene by an event specific molecular marker; and intercrossing at least two of the progeny plants which are multihomogenic for the transgene, to yield a population of plants which are multihomogenic for the transgene and express the desirable trait or traits tracing to the non-transgenic parent.

The method may further comprise backcrossing the population to one or more non-transgenic parent plants comprising germplasm encoding at least one desirable trait, to yield backcrossed progeny plants.

For example, the crossing of dihomogenic plants (AxxxByyy) to an elite null genotype will generate 25% dihomogenic progeny (AxxxByyy), which contain half the germplasm from the transgenic parent, and half the germplasm from the elite null parent. The dihomogenic segregants can be identified using event-specific molecular markers (described in detail elsewhere). An intercross of these dihomogenic plants will result in a population with 94.7% of the plants with the transgenic phenotype. Thus the current invention greatly decreases the time and resources required for the critical introgression of new germplasm into autopolyploid transgenic breeding programs.

According to another aspect of the present invention, trait stacking can be facilitated when one transgenic trait requires higher trait purity than the other. For example, in alfalfa the Roundup Ready™ trait may require varietal trait purity >95% (i.e. >95% of the plants in a variety having the RR phenotype). Other transgenic traits, such as insect resistance may require only 65-70% varietal trait purity to achieve the desired cultivar phenotype. In the latter case lower trait purity may be desired to help manage potential problems with the target insect developing resistance to the transgenic trait T imparting resistance. Crossing multihomogenic RR plants with transgenic plants with one or more copies of the T transgene at a single locus, using RR event specific PCR fingerprints and a PCR assay for transgene T, will allow the synthesis of populations with the trait purity combination described above (>95%RR and 70%T). The same outcome could be achieved by crossing plant(s) containing the RR transgene at one or more loci (e.g. locus A—RR_{A}) with a second plant(s) containing linked genetic loci containing both the RR transgene at an independent locus B₁ (RR_{B1}) and the one or more copies of the transgene T at a linked locus (T_{B2}). The resulting progeny would again be selected for multihomogenic individuals for the RR transgene using RR event specific PCR fingerprints and for the presence of the transgene T, by using T-specific PCR markers.

Prior to conducting crossing between independent events (e.g. A×B) and the implementation of PCR fingerprints to identify multihomogenic plants, a comprehensive characterization of the T₀ or BCₙ transgenic parent plants is required. Genomic DNA may be isolated from the T₀/BCₙ plants through the use of a variety of known techniques. Southern blot hybridization techniques can then be used to characterize the transgene integration. Information concerning the presence of a functional copy of the promoter, trait gene and transcriptional terminator can thus be obtained. For effective use of the technology described herein, T₀/BCₙ containing either a single copy insert or multiple complete copies of the transgene inserted at multiple independent loci would be required. The practice of these techniques and the interpretation of the data generated will be familiar to one skilled in the art of plant molecular genetics.

The present invention thus provides relatively straightforward methods of producing transgenic varieties of autopolyploid plants.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous inplementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Any autopolyploid plant can be the subject of the present invention. A preferred autopolyploid is alfalfa, an autotetraploid.

First, a comparison with the prior art will be provided. It is known that in an autopolyploid synthetic crop variety carrying transgenes from a single transgenic event, high trait purity (i.e. a high percentage of plants with the transgenic phenotype) requires parent plants in the duplex, triplex, or quadriplex condition for the transgene. One or more cycles of recurrent phenotypic selection and subsequent progeny testing are required to generate such genotypes given the simplex (Aaaa where A represents the transgene) condition of the T₀ or backcross (BCₙ) phenotype. For example, a single event transgenic plant subjected to three cycles of recurrent phenotypic selection for the transgenic phenotype would give rise to the following genotypes and genotypic frequencies:

| | |
|---|---|
| aaaa | 12% of the population (nulliplex for the transgene) |
| Aaaa | 35% of the population (monoplex for the transgene) |
| AAaa | 35% of the population (duplex for the transgene) |
| AAAa | 15% of the population (triplex for the transgene) |
| AAAA | 2% of the population (quadriplex for the transgene) |

Next, a single cycle of test crossing (A - - - x aaaa) to a susceptible tester is needed to identify the duplex, triplex and quadriplex individuals (52% of the total), both showing no susceptible segregants. Segregation ratios in progeny tests could be used to identify duplex vs triplex plants in the segregating progeny. A second cycle of test crossing and progeny testing (crossing the families from the first progeny test to a susceptible tester) is then needed to allow the identification of triplex (15% of the total) vs. quadriplex (2% of the total) individuals.

After identifying the quadriplex individuals in this way, they may be used in a random intercross to produce what is known as a first synthetic generation (Syn 1). In the Syn 1 generation, 100% of the plants will express the transgenic phenotype. However, in order to produce the transgenic plants on a larger, e.g., commercial, scale, subsequent synthetic generations are produced by further intercrossing. The progeny of these intercrosses are referred to as second synthetic generations (Syn 2), third synthetic generations (Syn 3), fourth synthetic generations (Syn 4), fifth synthetic generations (Syn 5), and subsequent later generations, and these generations will also have 100% expression of the transgenic phenotype.

As an alternative to using quadriplex individuals, a random intercross of triplex individuals results in a Syn 1 generation with 100% of the plants expressing the transgenic phenotype. If selection for the transgenic phenotype (e.g. herbicide resistance) is exercised prior to seed production of each Syn generation, the Syn 2 and Syn 3 generations will have 99.8% and 99.7% plants with the transgenic phenotype, respectively. Populations of the Syn 4, Syn 5, and later generations would also be expected to have very high trait purity.

As an alternative to using quadriplex or triplex individuals, a random intercross of duplex individuals results in a Syn 1 generation with 94.6% of the plants expressing the transgenic phenotype. If selection for the transgenic phenotype (e.g. herbicide resistance) is exercised prior to seed production of each Syn generation, the Syn 2 and Syn 3 generations will have 95.2% and 95.8% plants with the transgenic phenotype, respectively. Populations of the Syn 4, Syn 5, and later generations would also be expected to have the very high trait purity.

Typically, it is the Syn 3 generation that represents the commercial product when producing an autopolyploid crop. In this regard, it is highly desirable that a very high percentage of the plants produced in a Syn 3 generation exhibit the transgenic phenotype. However, the Syn 4, Syn 5, or later generations could be used for the production of a commercial autopolyploid crop.

Any of the above options, i.e., intercrossing plants with a single transgenic event in the duplex, triplex or quadriplex state, enables high levels (>95%) of expression of a transgenic phenotype in an autotetraploid population. However all three options require the use of progeny testing to identify genotypes containing more than one copy of the RR transgene at the single transgenic locus. A progeny testing program is resource intensive and will likely limit both the size of each progeny testing program (e.g. the number of duplex, triplex, or quadriplex individuals identified in the development of specific breeding populations) and the number of individual unique breeding populations developed with high transmission of the transgenic phenotype.

Moreover, the single event scenario risks genetic drift, or an unintentional skewing of the population away from the desired phenotype and narrows the genetic base of the resultant germplasm containing high transmission of the transgenic phenotype. This factor would likely have significant negative implications in the product development program of transgenic cultivars in autopolyploid plants.

In contrast, the multiple event transgenic system of the present invention uses multiple transgenic events combined with molecular markers (e.g. event specific PCR) to identify plants containing one or more copies of the transgene at multiple independent loci. Thus a relatively simple molecular assay, used to identify copies of the transgene at multiple loci, substitutes for the more resource intensive progeny testing, used to identify multiple copies of the transgene at a single locus. Both allow high transmission of the transgenic trait. The present invention does so in a manner that provides a significant savings in time and money in a product development program and with less associated risk in product performance.

In the early stages of development of transgenic varieties, multiple transgenic events are often being evaluated. A subset of the varieties would be submitted to the appropriate regulatory agencies for approval for commercial release. It is desirable that varieties approved for commercial release contain only the approved transgenic event(s). Compared with progeny testing, the use of genotypic selection, event specific PCR or similar techniques for identifying specific events, significantly lowers the probability of release of an unintended transgenic event. The present invention allows for event verification in the selection of parent plants prior to the production of Syn1 breeder seed.

In one illustrative embodiment of the present invention, multiple transgenic events A, B, C, D, etc. carried in separate transgenic plant lines, as follows:

Axxx (primary transgenic [T₀] or backcross progeny [BCₙ]) individual plants

Byyy (primary transgenic [T₀] or backcross progeny [BCₙ]) individual plants

Czzz (primary transgenic [T₀] or backcross progeny [BCₙ] individual plants

Dwww (primary transgenic [T₀] or backcross progeny [BCₙ]) individual plants

A, B, C, and D represent transgenes incorporated into the genomes of distinct transgenic plant lines, leading, in one illustrative embodiment, to essentially the same phenotype when expressed in the plants. They may be identical transgenes, or alternatively, they may contain some differences in the regulatory (e.g. promoter) regions and/or in the coding or non-coding portions of the transgene (e.g. trait gene). Because A, B, C and D represent independent transgenic events, the transgenes in these transgenic lines are incorporated in the plant DNA at different loci within the genome. Consequently, the transgenes from A, B, C and D act as separate genes and therefore segregate independently.

Therefore, in one illustrative embodiment, the multiple transgenic events A, B, C, and D may represent identical transgenes that are incorporated into different parts of a plant species genome. In a further embodiment, A, B, C and D may represent transgenes containing essentially the same coding regions, but having differences in promoter or other regulatory regions of the transgene. In yet a further embodiment, A, B, C and D may represent transgenes with the same or similar promoters and/or other regulatory regions but that contain certain differences in their coding regions. For example, some of the coding regions may contain certain modifications that confer enhanced expression or some other advantage not offered by an unmodified version of the coding region. Alternatively, the coding regions may be entirely unrelated by homology, but still give rise to the same or substantially similar phenotype traits, possibly by distinct mechanisms. These and other variations on this theme will be recognized by the skilled individual to be clearly within the scope of the present invention.

In one illustrative embodiment of the present invention, single transgenic events A and B are used for the production of dihomogenic individuals that can be used in the efficient development of commercial cultivars of transgenic autopolyploid plants with very high transmission of the transgenic phenotype.

Dihomogenic plants are produced from a cross between parents with independent transgenic events (e.g. A and B, as described above). These parents trace to T₀ or BCₙ progeny that are simplex for the transgene, or plants/populations derived therefrom. Dihomogenic plants are identified from the segregating [(Axxx)×(Byyy)] progeny using event specific PCR or a similar technique, as will be described in more detail below.

In another illustrative embodiment of the present invention, single transgenic events A, B, and C are used for the production of trihomogenic individuals that can be used in the efficient development of commercial cultivars of transgenic autopolyploid plants with very high transmission of the transgenic phenotype.

Trihomogenic plants are produced from a cross between a dihomogenic parent (e.g. AxxxByyy, as described above) and a second plant/population containing a third independent transgenic event (C, D, etc. as described above). These parents trace to independent T₀ or BCₙ progeny that are simplex for the transgene, or plants/populations derived therefrom. Trihomogenic plants are identified from the segregating [(AxxxByyy)×(Czzz)] progeny using event specific PCR or a similar technique, as will be described in more detail below.

In another illustrative embodiment of the present invention, single transgenic events A, B, C and D are used for the production of tetrahomogenic individuals that can be use in the efficient development of commercial cultivars of transgenic autopolyploid plants with very high transmission of the transgenic phenotype.

Tetrahomogenic plants can be produced from a cross between dihomogenic parents, each containing copies of the transgene at two unique and independent loci (e.g. AxxxByyy and CzzzDwww, as described above). These parents trace to independent T₀ or BCₙ progeny that are simplex for the transgene, or plants/populations derived therefrom. Tetrahomogenic plants are identified from the segregating [(AxxxByyy)×(CzzzDwww)] progeny using event specific PCR or a similar technique, as will be described in more detail below.

The following diagram further illustrates this embodiment:

Axxxyyyyzzzzwwww×xxxxByyyzzzzwwww→AxxxByyyzzzzwwww (dihomogenic for A and B, identified by event specific PCR) and

xxxxyyyyczzzwwww×xxxxyyyyzzzzDwww→xxxxyyyyCzzzDwww (dihomogenic for C and D, identified by event specific PCR)

A double cross of the identified dihomogenic plants can then be used to produce “1,1,1,1 tetrahomogenic” plants containing the transgene (in the simplex condition) at four independent loci:

AxxxByyyzzzwwww×xxxxyyyyCzzzDwww→AxxxByyyCzzzDwww (tetrahomogenic for A, B, C, and D, identified by event specific PCR)

Mathematical models have been developed to predict trait purity (i.e. % of plants in a population with the transgenic phenotype) of transgenic traits in an autotetraploid population. The table below shows expected trait purity in the Syn1, Syn2 and Syn3 generations for several monogenic and multigenic population types, developed using various selection methods. The selection method PRSN refers to n cycles of phenotypic recurrent selection; the selection method GRSN refers to n cycles of genotypic recurrent selection where a molecular marker (e.g. event specific PCR) is used to identify multihomogenic plants; and the selection method PTₙ refers to n cycles of genotypic recurrent selection where progeny testing is used to identify a particular genotype (e.g. duplex, triplex or quadriplex). Roundup Ready™ is used, purely by way of example and not to be construed as limiting the invention in any way, as an example transgenic trait for this model with the assumption that there will be selection for Roundup tolerance during the production of Syn 1, Syn 2, Syn 3, Syn 4, Syn 5, or later generation seed.

TABLE 2
Expected trait purity of autotetraploid
populations developed using one to four
transgenic events and following various selection schemes

| | | % RR progeny | | |
|---|---|---|---|---|
| | | (w/phenotypic selection | | |
| | | each Syn generation) | | |
| Selection | Genotype | Syn 1 | Syn 2 | Syn 3 |
| PRS₁PT₁ | Duplex | 93.7 | 94.6 | 95.4 |
| PRS₃PT₁ | Triplex | 100.0 | 99.6 | 99.4 |
| GRS₁ | Dihomogenic | 93.7 | 92.7 | 93.8 |
| PRS₁GRS₁ | Dihomogenic | 95.3 | 94.5 | 95.3 |
| PRS₃GRS₁ | Dihomogenic | 96.8 | 96.3 | 96.6 |
| GRS₂ | Trihomogenic | 98.4 | ** | ** |
| GRS₂ | Tetrahomogenic | 99.6 | ** | ** |

**models have not been developed to predict genotypic arrays for advanced generations in these more complicated three and four gene systems. However, predictions from the two gene system suggest that multihomogenic populations with very high trait purity experience only very small changes in % RR progeny from Syn1 to Syn3 generations.

Trait purity of multihomogenic lines can be enhanced by phenotypic recurrent selection within single event lines prior to the intercrossing of these lines to generate multihomogenic plants. This is illustrated in Table 2 in the comparison between GRS₁ and PRS₁GRS₁. Note that additional cycles of recurrent selection prior to the intercrossing of events (e.g. PRS₃GRS₁, etc.) will result in further increases in trait purity. In one embodiment of the present invention, one or more cycles of phenotypic recurrent selection is used to increase transgene frequency in single event lines prior to the crossing to generate multihomogenic plants. Note also from Table 2 that trait purity levels above 96% can only be achieved with multihomogenic lines (dihomogenic with PRS, trihomogenic or tetrahomogenic, etc.) or monohomogenic lines in the triplex or quadriplex condition.

Transgenic progeny that are either dihomogenic, trihomogenic or tetrahomogenic for transgenes A, B, C and/or D can be readily identified using conventional molecular biological techniques. In one illustrative embodiment, the progeny of the above intercrosses are screened using the polymerase chain reaction (PCR).

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates are added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. Many polymerase chain reaction methodologies have been developed for various applications and are well known in the art (Sambrook et al., 1989).

Because each transgenic event A, B, C and D is associated with integration of a transgene in unique regions of the plant genome, one illustrative embodiment of the invention employs a rapid PCR-based approach for the identification of the monohomogenic, dihomogenic, trihomogenic and tetrahomogenic individuals resulting from the above intercrosses. This approach requires only the characterization of a small amount of the flanking genomic nucleotide sequence of the region of the plant genome immediately upstream (5′) and/or downstream (3′) of the transgene integration site. A variety of well known molecular techniques can be used to clone and characterize these regions. For example, “gene walker” technology (commercially available from Clontech, Palo Alto, Calif.) uses a PCR based technology for walking along genomic DNA. This and other techniques may routinely be employed by those trained in molecular biology.

The nucleotide sequence of these flanking regions can then be determined using standard DNA sequencing techniques. This flanking DNA sequence information, and sequence information internal to the transgene itself, are used to design polymerase chain reaction (PCR) primers such that one primer hybridizes with a nucleotide sequence within the 5′ or 3′ region of the transgene, while the other primer sequence hybridizes with flanking region of the plant genome. Having the necessary DNA sequence information at hand, the skilled person in the art will be quite familiar with the design and use of PCR primers. Briefly, and for purposes of illustration only, the length of the PCR primers is generally 20-30 bases, allowing for the formation of duplex molecules that are both stable and specific.

Once primer sequences for each transgenic event A, B, C, D, etc. have been identified, tested and optimized, PCR amplification of plant genomic DNA taken from progeny of the intercrosses will provide a highly sensitive event-specific PCR fingerprint for each transgenic event. Moreover, by designing PCR primer pairs for different integration events with compatible annealing temperatures, but yielding different length amplification products, one can amplify multiple products for dihomogenic, trihomogenic and tetrahomogenic plants in the same PCR reaction by multiplexing the reactions. This procedure will thus identify individual plants resulting from the above intercrosses containing 1 or more copies of the transgene at independent loci A, B, C and/or D.

Other techniques for identifying transgenic plants, such as single nucleotide polymorphism (SNP) technologies, Invader OS (commercially available from Third Wave Technologies Inc., Madison, Wis.), or other techniques for detecting single base changes or unique sequences known to one of ordinary skill in the art, may be used instead of or in addition to event-specific PCR.

### Transgenic Plants

The present invention relates generally to autopolyploid transgenic plants, and, in many illustrative embodiments, to autotetraploid transgenic plants, such as alfalfa. As used herein, the term “transgenic plants” is intended to refer to plants, the genome of which has been augmented by at least one incorporated DNA sequence, also referred to herein as a genetic construct or a recombinant plant transgene. Such DNA sequences can include any gene, gene fragment or DNA sequence that one desires to introduce into plant species. Generally, but not always, the introduced DNA sequences will be DNA sequences not normally present in the plant species but which one desires to express in the plant to achieve certain beneficial traits not normally found in the species.

Exemplary DNA sequences that have been introduced into plants include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to DNA sequences or genes which are not normally present in the cell being transformed, or perhaps are simply not present in the form, structure, etc., as found in the transforming DNA segment or gene, or genes which are normally present yet which one desires to alter functionally, e.g., to have overexpressed. Thus, the term “exogenous” gene or DNA refers to any gene or DNA segment that is introduced into a recipient cell, regardless of whether a similar gene may already be present in such a cell. “Introduced”, or “augmented” in this context, is known in the art to mean introduced or augmented by the hand of man.

### Plant Phenotype Modification

As is well known within the art of plant genetic engineering, numerous possibilities exist for the production of transgenic plants having desired phenotypes. It is important to note, however, that the present invention is in no way limited by the particular transgene(s) employed or the manner in which the transgenes are introduced into plant cells to produce the transgenic plants (e.g. Agrobacterium mediated transformation, biolistics, electroporation, etc.). In this regard, the following examples of transgenic plant traits are provided for purposes of illustration only. The transgene used for achieving a desired transgenic trait will often be genes that direct the expression of a particular protein or polypeptide product, but they may also be non-expressible DNA segments, e.g., transposons such as Ds that do not direct their own transposition. As used herein, an “expressible gene” is any gene that is capable of being transcribed into RNA (e.g., mRNA, antisense RNA, etc.) or translated into a protein, expressed as a trait of interest, or the like, etc., and is not limited to selectable, screenable or non-selectable marker genes.

The choice of the particular recombinant DNA sequences to be incorporated into recipient plant cells will often depend on the purpose of the transformation. One of the major purposes of transformation of crop plants is to add some commercially desirable, agronomically important traits to the plant. Such traits include, but are not limited to, herbicide resistance or tolerance; insect resistance or tolerance; disease resistance or tolerance (viral, bacterial, fungal, nematode); stress tolerance or resistance, as exemplified by resistance or tolerance to drought, heat, chilling, freezing, excessive moisture, salt stress; oxidative stress; increased yields; chemical composition; physical appearance; male sterility; drydown; standability; prolificacy; starch properties; fiber, protein, or oil quantity or quality; and the like. In one embodiment, the expression of an EPSPS or GOX coding region may impart resistance or tolerance to glyphosate herbicides. One may desire to incorporate one or more genes conferring any such desirable trait or traits.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Populations of Roundup-tolerant alfalfa plants were derived from F1 crosses between populations of fall dormancy plants that had a single copy of an independent RR transgene event (e.g. Byyy×Czzz). Approximately two weeks after germination, seedling progeny from the F1 cross were sprayed with Roundup Ultra and tolerance values were obtained (F1% RR). Event specific PCR was used as a molecular marker to identify GRS1 Syn0 plants from the F1 population. These are plants that carry both events (i.e. dihomogenic plants). The plants identified as being dihomogenic were intercrossed and the Roundup tolerance in the progeny (GRS1 Syn1 population) was determined. Results obtained with five event combinations are presented, and the values predicted from modeling are also given. The observed values are substantially as predicted.

TABLE 3
Experimental Roundup tolerance and event specific PCR data for
dormant alfalfa populations using a two event breeding scheme for one
cycle of genotypic recurrent selection

| | F1 | GRS1 Syn0 | GRS1 Syn1 |
|---|---|---|---|
| F1 cross | % RR | % dihomogenic | % RR |
| B × C | 72.3 | 34.3 | 93.8 |
| B × D | 67.2 | 32.2 | 93.7 |
| B × G | 73.0 | 31.2 | 94.6 |
| C × D | 72.8 | 33.3 | 92.8 |
| C × G | 69.6 | 33.1 | 95.8 |
| Predicted value | 75.0 | 33.3 | 93.7 |

### Example 2

Populations of Roundup-tolerant alfalfa plants were derived from F1 crosses between populations of non-dormant plants that had a single copy of an independent RR transgene event (e.g. Byyy×Czzz). Approximately two weeks after germination, seedling progeny from the F1 cross were sprayed with Roundup Ultra and Roundup tolerance values were obtained (F1% RR). Event specific PCR was used as a molecular marker to identify GRS1 Syn0 plants that carry both events (i.e. dihomogenic plants). The plants identified as being dihomogenic were intercrossed and the Roundup tolerance of the progeny (GRS1 Syn1 population) was determined. Event specific PCR was used as a molecular marker to identify dihomogenic plants in the GRS1 Syn1 populations. These plants (GRS2 Syn0) were intercrossed and Roundup tolerance of the progeny (GRS2 Syn1 population) was determined. Results obtained with three event combinations are presented. The values predicted by modeling are also presented. The observed values are substantially as predicted.

TABLE 4
Experimental Roundup tolerance and event specific PCR data for non-dormant
alfalfa populations using a two event-breeding scheme for two cycles of genotypic
recurrent selection

| | | GRS1 Syn0 | GRS1 Syn1 | GRS2 Syn0 | GRS2 Syn1 |
|---|---|---|---|---|---|
| F1 cross | F1 % RR | % dihomgenic | % RR | % dihomogenic | % RR |
| B × G | 75.7 | 35.6 | 87.9 | 58.0 | 96.5 |
| C × D | 75.6 | 31.5 | 91.5 | 57.2 | 95.0 |
| D × G | 76.2 | 31.6 | 92.3 | 59.1 | 95.4 |
| Predicted value | 75.0 | 33.3 | 93.7 | 60.1 | 97.0 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

### Patent Publications

U.S. Pat. No. 4,683,195

U.S. Pat. No. 4,683,202

U.S. Pat. No. 4,800,159

U.S. Pat. No. 5,324,253

U.S. Pat. No. 5,405,765

U.S. Pat. No. 5,463,175

U.S. Pat. No. 5,484,956

U.S. Pat. No. 5,633,435

U.S. Pat. No. 5,731,202

U.S. Pat. No. 5,886,244

Int. Pat. Appl. Publ. No. WO 84/02913

### Scientific Publications

Barkai-Golan, R., Mirelman, D., Sharon, N. (1978) Arch. Microbio 116:119-124.

Bemal-Lugo and Leopold, A. C. 1992. Plant Physiol. 98:1207-1210.

Blackman, S. A., Obendorf, R. L., Leopold, A. C. 1992. Plant Physiol. 100:225-230

Bowler, C. van Montagu, M., and Inze, D. 1992. Ann Rev. Plant Physiol. 43:83-116

Broakaert, W. F., Parijs, J., Leyns, F., Joos, H., Peumans, W. J. (1989) Science 245:1100-1102.

Campbell, W. C., Ed., 1989. Avermectin and Abamectin

Coxson, D. S., McIntyre, D. D., and Vogel, H. J. 1992. Biotropica 24:121-133.

Cristou P., McCabe D. E., Swain W. F. (1988). Plant Physio 87:671-674.

Cutler, A. J., Saleem, M., Kendell, E., Gusta, L. V., Georges, F., Fletcher, G. L. (1989), J Plant Physiol 135:351-354.

Czapla & Lang (1990), J. Econ. Entomol. 83:2480-2485.

Desgagnes et al. (1995) Plant Cell Tissue Organ Culture 42:129-140.

Dunn, G. M. et al., (1981) Can. J. Plant Sci., 61:583:593.

Eichholtz, D. A., Rogers, S. G., Horsch, R. B., Klee, H. J., Hayford, M., Hoffman, N. L. Bradford, S. B., Flink, C., Flick, J., O'Connell, K. M., Frayley, R. T. 1987. Somatic Cell Mol. Genet. 13:67-76.

Erdmann, N., Fulda, S., and Hagemann, M. 1992. J. Gen. Microbiology 138:363-368.

Fraley et al., Bio/Technology, 3:629-635, 1985

Guerrero, F. D., Jones, J. T., Mullet, J. E. 1990. Plant Molecular Biology 15:11-16.

Gupta, A. S., Heinen, J. L., Holaday, A. S., Burke, J. J., and Allen, R. D. 1993. Proc. Natl. Acad. Sci USA 90:1629-1633.

Hammock, B. D., Bonning, B. C., Possee, R. D., Hanzlik, T. N., and Maeda, S. (1990), Nature 344:458-461.

Hilder, V. A., Gatehouse, A. M. R., Sheerman, S. E., Barker, R. F., Boulter, D. (1987) Nature 330:160-163.

Ikeda, H., Kotaki, H., Omura, S. (1987), J. Bacteriol 169:5615:5621.

Johnson, R., Norvdez, J., An, G, and Ryan, C. (1989), proc. Natl. Acad. Sci. USA 86:9871-9875.

Jorgensen et al., Mol. Gen. Genet., 207:471, 1987.

Kaasen, I., Falkenberg, P., Styrvold, O. B., Strom, A. R. 1992. J. Bacteriology 174:889-898.

Karsten, U., West, J. A. and Zuccarello, G. 1992. Botanica Marina 35:11-19.

Klee et al., Bio/Technology, 3:637-642, 1985.

Klein, T. M., Gradziel, T., Fromm, M. E., Sanford, J. C. 1988. Bio/Technnology 6:559-563

Koster, K. L. and Leopold, A. C. 1988. Plant Physiol. 88:829-832.

Kozak, M. 1984. Nucl Acids Res 12:857-872.

Lee and Saier, 1983 J. of Bacteriol. 153-685.

Loomis, S. H. carpenter, J. F., Anchordoguy, T. J. Crowe, J. H., and Branchini, B. R. 1989. J. Expt. Zoology 252:9-15.

Murdock et al., 1990 Phytochemistry 29:85-89.

Napoli, C., Lenieux, C., Jorgense, R. 1990. Plant Cell 2:279-289.

Reed, R. H., Richardson, D. L., Warr, S. R. C., Stewart, W. D. P. 1984. J. Gen. Microbiology 130:1-4.

Rogers et al., In: Methods For Plant Molecular Biology, A. Weissbach and H. Weissbach, eds., Academic Press Inc., San Diego, Calif. 1988.

Sambrook, J. Fritsch, E. F., and Maniatis, T. (1989), Molecular Cloning, A Laboratory Manual End. 2d.

Shagan, T., Bar-Zvi, D. 1993. Plant Physiol. 101:1397-1398.

Smith, C. J. S., Watson, C. F., Bird, C. R., Ray, J., Schuch, W. and Grierson, D. 1990. Mol. Gen. Genet. 224:447-481.

Spielmann et al., Mol. Gen. Genet., 205:34, 1986.

van der Krol, A. R., Mur, L. A., Beld, M., Mol, J. N. M. 1990. Plant Cell 2:291-299.

Velten and Schell, “Selection-expression plasmid vectors for use in genetic transformation of higher plants,” Nucl. Acids Res., 13(19):6981-6998, 1985.

Velten et al., EMBO J., 3:2723 -2730, 1984.

Vernon, D. M. and Bohnert, H. J. 1992. The EMBO J. 11:2077-2085.

Watrud, L. S., Perlak, F. J. Tran, M. T., Kusano, K., Mayer, E. J., Miller-Wildemann, M. A., Obukowicz, M. G., Nelson, D. R., Kreitinger, J. P., and Kaufnan, R. J. (1985), in Engineered Organisms and Environment, H. O. Halvorson et a., eds., Am. Soc. Microbiol., Washington, D.C.

Wolter, F., Schmidt, R., and Heinz, E. 1992. The EMBO J. 4685-4692.

Wyn-Jones, R. G., and Storey, R. 1981 Physiology and Biochemistry of Drought Resistance in Plants: 171-204.

What is claimed:

1. A method for achieving high levels of transmission of a transgenic trait in an autopolyploid crop, comprising:
providing multiple independent autopolyploid transgenic plant lines, wherein each of said plant lines comprises at least one recombinant transgene incorporated into a different region of the plant genome, and each of said plant lines exhibits a transgenic trait attributable to said transgene;
intercrossing plants from the independent transgenic plant lines for one or more generations to obtain progeny plants that are multihomogenic for the recombinant transgene;
identifying said multihomogenic plants by an event specific molecular marker; and
intercrossing said progeny plants to produce at least a first synthetic generation of plants.

2. The method of claim 1, wherein the identifying step comprises using event specific polymerase chain reaction (PCR).

3. The method of claim 1, wherein greater than 90% of the first synthetic generation of plants exhibit the transgenic trait.

4. The method of claim 1, further comprising intercrossing the first synthetic generation of plants to produce a second synthetic generation of plants.

5. The method of claim 4, wherein greater than 90% of the progeny of the second synthetic generation of plants exhibit the transgenic trait.

6. The method of claim 4, further comprising intercrossing the second synthetic generation to produce a third synthetic generation of transgenic plants.

7. The method of claim 6, wherein greater than 90% of the progeny of the third synthetic generation of plants exhibit the transgenic trait.

8. The method of claim 6, further comprising intercrossing the third or later synthetic generation to produce a fourth or later synthetic generation of transgenic plants.

9. The method of claim 8, wherein greater than 90% of the progeny of the fourth or later synthetic generation of plants exhibit the transgenic trait.

10. The method of claim 1, further comprising crossing said multihomogenic plants with a second transgenic plant line, wherein the second transgenic plant line comprises a second recombinant transgene incorporated into a single region of the plant genome, and the second transgenic plant line exhibits a transgenic trait attributable to said second recombinant transgene.

11. The method of claim 1, wherein at least one of the multiple independent autopolyploid transgenic plant lines comprises a second recombinant transgene, wherein the second recombinant transgene is at a locus linked to the region of the genome of said plant line into which the first recombinant transgene is incorporated.

12. A method for obtaining a dihomogenic transgenic alfalfa plant line, comprising:
providing two transgenic alfalfa plant lines, each of said plant lines having a recombinant transgene incorporated into a different region of the alfalfa genome;
intercrossing said two transgenic alfalfa plant lines to produce a first set of progeny; and
identifying the dihomogenic transgenic plant line from said first set of progeny.

13. The method of claim 12, wherein the identifying step comprises using event specific polymerase chain reaction (PCR).

14. The method of claim 12, further comprising intercrossing said dihomogenic transgenic plant line with a third transgenic alfalfa plant line, said third transgenic alfalfa plant line having a recombinant transgene incorporated into a different region of the alfalfa genome, to produce a second set of progeny; and identifying a trihomogenic transgenic plant line from said second set of progeny.

15. The method of claim 12, further comprising intercrossing a third and a fourth transgenic alfalfa plant line, each of said plant lines having a recombinant transgene incorporated into a different region of the alfalfa genome, to produce a second set of progeny; identifying a second dihomogenic transgenic plant line from the second set of progeny; intercrossing the dihomogenic transgenic plant line and the second dihomogenic transgenic plant line, to produce a third set of progeny; and identifying a tetrahomogenic transgenic plant line from the third set of progeny.

16. A method for achieving high levels of transmission of a transgenic trait in a synthetic alfalfa generation, comprising:
providing a plurality of dihomogenic, trihomogenic, or tetrahomogenic transgenic alfalfa plants;
intercrossing said plurality of dihomogenic, trihomogenic, or tetrahomogenic transgenic plants to produce a first synthetic generation of plants; and
intercrossing the first synthetic generation of plants to produce a second synthetic generation of plants.

17. The method of claim 16, wherein greater than 95% of the second synthetic generation of plants exhibits the transgenic trait.

18. The method of claim 16, further comprising intercrossing the second synthetic generation of plants to produce a third synthetic generation of plants.

19. The method of claim 18, wherein greater than 95% of the third synthetic generation of plants exhibits the transgenic trait.

20. The method of claim 18, further comprising intercrossing the third or a later synthetic generation of plants to produce a fourth or later synthetic generation of plants.

21. The method of claim 20, wherein greater than 95% of the fourth or later synthetic generation of plants exhibits the transgenic trait.

22. An autopolyploid transgenic plant, comprising two recombinant transgenes, each of said transgenes incorporated into a different region of the plant genome.

23. The transgenic plant of claim 22, wherein each of said transgenes causes essentially the same transgenic trait when expressed in said transgenic plant.

24. The transgenic plant of claim 23, wherein said transgenic trait is selected from insect resistance or herbicide resistance.

25. The transgenic plant of claim 22, further comprising a third recombinant transgene, said transgene being incorporated into a different region of the plant genome than the other two transgenes.

26. The transgenic plant of claim 25, wherein each of said transgenes causes essentially the same transgenic trait when expressed in said transgenic plant.

27. The transgenic plant of claim 25, wherein said transgenic trait is selected from insect resistance or herbicide resistance.

28. The transgenic plant of claim 25, further comprising a fourth recombinant transgene, said transgene being incorporated into a different region of the plant genome than the other three transgenes.

29. The transgenic plant of claim 28, wherein each of said transgenes causes essentially the same transgenic trait when expressed in said transgenic plant.

30. The transgenic plant of claim 29, wherein said transgenic trait is selected from the group consisting of insect resistance and herbicide resistance.

31. The transgenic plant of claim 22, wherein each said recombinant transgene comprises a promoter and a coding region.

32. The transgenic plant of claim 31, wherein said coding region comprises an EPSPS coding region or a GOX coding region.

33. The transgenic plant of claim 31, wherein the promoter of each of said transgenes is the same.

34. The transgenic plant of claim 31, wherein the promoter of each of said transgenes is different.

35. The transgenic plant of claim 31, wherein the coding region of each of said transgenes is the same.

36. The transgenic plant of claim 31, wherein the coding region of each of said transgenes is different.

37. The transgenic plant of claim 31, wherein the promoter of each said transgene is the same and the coding region of each said transgene is the same.

38. The transgenic plant of claim 31, wherein the plant is an autotetraploid plant.

39. The transgenic plant of claim 31, wherein the plant is an alfalfa plant.

40. A transgenic seed obtained from the plant of claim 22.

41. A transgenic alfalfa plant, comprising at least two recombinant transgenes, wherein each of said transgenes is capable of segregating independently of the other transgenes, and each of said transgenes causes essentially the same phenotype when expressed in said plant.

42. The transgenic alfalfa plant of claim 41, comprising three recombinant transgenes, each of said transgenes is capable of segregating independently of the other transgenes, wherein each of said transgenes when expressed in said plant causes essentially the same phenotype.

43. The transgenic alfalfa plant of claim 41, comprising four recombinant transgenes, wherein each of said transgenes is capable of segregating independently of the other transgenes, and each of said transgenes when expressed in said plant causes essentially the same phenotype.

44. A method for introgressing non-transgenic germplasm into a transgenic autopolyploid crop, comprising:
providing one or more donor parents that are multihomogenic for the transgene;
crossing the one or more donor parents to one or more non-transgenic parent plants comprising germplasm containing at least one desirable trait not present in the donor parent, to yield progeny plants, wherein at least one of the progeny plants is multihomogenic for the transgene;
identifying progeny plants which are multihomogenic for the transgene by an event specific molecular marker; and
intercrossing at least two of the progeny plants which are multihomogenic for the transgene, to yield a population of plants which are multihomogenic for the transgene and express the desirable trait or traits tracing to the non-transgenic parent.

45. The method of claim 44, further comprising backcrossing the population to one or more non-transgenic parent plants comprising germplasm containing at least one desirable trait not present in the donor parent, to yield backcrossed progeny plants.
